(19) United States
(12) Patent Application Publication
(10) Pub. No.: US 2008/0108924 A1
(43) Pub. Date: May 8, 2008

(54) Wound Dressing

(76) Inventor: Peter Molan, Hamilton (NZ)

Correspondence Address: ABELMAN, FRAYNE & SCHWAB, 666 THIRD AVENUE, 10TH FLOOR, NEW YORK, NY, 10017, US

(21) Appl. No.: 11/597,146

(22) PCT Filed: May 20, 2005
(86) PCT No.: PCT/NZ05/00104

§ 371 Date: Dec. 3, 2007

(30) Foreign Application Priority Data
May 21, 2004 (NZ) 533121

(51) Int. Cl. A61F 13/00

(52) U.S. Cl. 602/48; 602/42

(57) ABSTRACT

A wound dressing (1) including at least two adhesive portions (2) capable of adhering to and being removed from skin, and at least two non-elastic threads (3) held between the adhesive portions, characterised in that the spacing between the non-elastic threads allows direct access to the wound or skin between the adhesive portions.

### TECHNICAL FIELD

This invention relates to a wound dressing. Specifically, this invention relates to a dressing for the closure of wounds without use of sutures.

### BACKGROUND ART

Wounds above a certain size, whether created by accident or by surgical techniques, must be held closed in order to reduce bleeding and promote healing, while reducing subsequent scarring.

Common methods of wound closure methods typically involve the use of sutures, staples or so forth, whereupon the sides of the wound are held together by threads or other foreign bodies inserted into the skin.

However, whilst good at holding the wound closed, sutures are generally uncomfortable as they pull on the tissue surrounding the wound when the skin is stretched in any direction.

Inserting foreign bodies into the skin usually requires either local or general anaesthetic to reduce the discomfort to the patient and thus allow the sutures to be properly placed.

The requirement for anaesthetics means suturing cannot be readily carried out in the field, where first aid may be some distance off.

Further, sutures typically can only be placed by trained personnel, where much practice is needed to place sutures quickly whilst attempting to reduce future scarring.

As a wound heals, the skin surrounding the wound generally begins to grow around the suture thread. As such, when the time comes to remove the sutures, their removal is often painful and pulls on the scar tissue.

The tissue is also likely to react to foreign bodies in the skin, often resulting in a further degree of scarring.

While many methods of suturing exist, it is typically very difficult to align both sides of the wound without bunching of the skin and thus the formation of a scar. This is particularly applicable when sutures are used to close uneven or jagged wounds.

The necessity to remove the sutures or staples before they are surrounded by tissue or before a severe foreign body reaction develops often necessitates sutures being removed before the wound has properly healed.

Typically, a wound may not reach full strength until six months after injury, whereas sutures are generally removed within two weeks. As such, the scar may reopen or stretch, becoming more visible.

A number of wound dressings exist for smaller wounds that do not require the use of sutures; such as sticking plasters, butterfly closures and so forth. However, these are designed only for small cuts and are not suitable for use with larger wounds.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a wound dressing including:

at least two adhesive portions capable of adhering to and being removed from skin, and

at least two non-elastic threads held between the adhesive portions,

characterised in that

the spacing between the non-elastic threads is sufficient to allow direct access to a wound or skin between the adhesive portions.

According to another aspect of the present invention there is provided a method of treating a wound using a wound dressing,

characterised by the steps of
a) applying an adhesive portion of the wound dressing to skin,
b) pushing the wound edges together, and
c) adhering a second adhesive portion of the wound dressing to the skin to hold the wound together, and
d) maintain tension across the wound.

Preferably, the threads are positioned with respect to the adhesive portions so that tension can be maintained between the adhesive portions by the threads when the dressing is applied to the skin.

The adhesive portions are preferably adhesive strips known and used in the art in wound dressings, and in preferred embodiments may be placed on either side of a wound.

In preferred embodiments of the present invention, there will be at least two adhesive portions capable of adhering to and being removed from the skin.

This should not be seen as a limitation on the present invention however, for example a ring of adhesive may be utilised to adhere to the dressing to the wound/skin region or any number of adhesive portions may be placed either side of the wound and could conceivably be used with the present invention.

Preferably, the adhesive portions will also contain elastic threads to allow the adhesive portions to stretch in a direction substantially parallel to that of the wound. This will allow comfortable movement of the skin whilst maintaining tension across the wound.

In one embodiment the adhesive portions can be made from elastomeric material sold under the trade mark Hypafix™, manufactured by BSN Medical GmbH & Co. of KG, Hamburg, Germany.

This should not be seen as a limitation on the present invention as a liquid adhesive applied just before placing on the skin may be utilised, for example a cyanomethacrylate adhesive such as “superglue”.

In other embodiments, a removable protective cover is used for the adhesive portions to preserve the adhesive portion of the strip.

In preferred embodiments of the present invention, the non-elastic threads may be standard monofilament suture threads as known and used in the art, such as nylon, polypropylene and so forth.

This should not be seen as a limitation on the present invention however for any type of non-elastic thread could conceivably be used with the present invention.

For example, silk may be used, a material not recommended for standard suturing as it causes too much tissue reaction.

In preferred embodiments of the present invention, the number of non-elastic threads required for the dressing will vary depending upon the size of the threads and/or dressing size and is not limited to a specified number of threads.

Preferably, the non-elastic thread will be fused or bonded to the adhesive portion.

However, once again this should not be seen as a limitation on the present invention for the threads may be woven into the adhesive portions or attached by some other means.

The non-elastic threads are preferably spaced sufficiently far apart to allow access to the wound without needing to disturb the adhesive portions.

Preferably the non-elastic threads are spaced in the order of 5 mm apart from each other. This is one of the optimum spacing distances required to maintain tension and enable direct contact with the wound.

However, it should be appreciated this is given by way of example only, as the spacing is likely to be within a range of 1-10 mm.

While a spacing of greater than 10 mm could be used, the ability of the wound dressing to hold the wound closed could be compromised.

The term “access” should be taken to mean the ability to apply or change a treatment composition to the wound without having to disturb the rest of the dressing and to examine the wound when necessary.

The treatment composition may be any standard anti-bacterial or healing composition known in the art, in the form of an ointment, cream, gel, solid or so forth and may be able to be continually applied without having to disturb the rest of the dressing.

The treatment composition may be in the form of a removable system able to be applied to the wound without having to disturb the rest of the dressing.

The treatment composition can be configured with the wound dressing by incorporating into a pad or the pad itself.

The treatment composition where removable may be held in place by adhesive portions or attached by some other means, for example Velcro.

In preferred embodiments of the present invention the treatment composition is a gelled honey composition. Gelled honey compositions have been previously described by the applicant (for example WO 01/41776) which overcome the difficulties in localising and maintaining honey to the required area of treatment. It is envisaged these compositions could be used in conjunction with the present invention.

The honey composition could be in the form of an ointment or salve, or may be in the form of a formable, pliable solid.

In some preferred embodiments of the invention, the gelled honey composition will be incorporated into the wound dressing during manufacture.

In other preferred embodiments the honey composition may be separate to the wound dressing and be applied to the wound at substantially the same time as the dressing.

It is also anticipated such compositions will slowly dissolve in body fluids, allowing the treatment composition to be readily changed without disturbing the wound dressing.

Further, the honey compositions preferably may be readily dissolved from the dressing using solvents such as water or saline, to inspect the wound or change the composition.

The anti-bacterial and healing properties of honey are well known, with recent international medical literature showing honey as being effective in treating wounds, burns and skin ulcers.

Recorded observations include that inflammation, swelling and pain are quickly reduced, that sloughing of necrotic tissue occurs without the need for debridement, and that growth of tissues to repair the wound is stimulated. As a consequence, healing is able to occur rapidly and with minimal scarring.

Further, as healing processes will not usually occur unless infection is cleared from a wound, the antibacterial properties of honey enable this to occur without the use of other antibacterial agents which often cause a degree of tissue irritation and thus may actually slow the healing process.

Although honey is preferably used as a whole, the active components of honey may be extracted from honey or other natural products, artificially synthesised or otherwise modified and used either individually or in combination.

The composition may further include other beneficial compounds, such as pharmaceuticals, vitamins, hormones, cations, plant extracts and so forth to assist in wound healing. It is anticipated the benefit of such compounds in wound healing would be readily understood by a person skilled in the art, for example the use of cations in assisting blood clotting.

Other modifications may include means for reducing free moisture such as wound exudates, ensuring the area under a dressing remains moist but not wet. This may include the addition of water absorbing components such as alginate and/or water removing components, which remove water by chemical reaction.

A further benefit of honey compositions is that they soften at more elevated temperatures. In many instances, while it is desirable that the composition remains non-running at body temperature, the flexibility of gelled honey strips enables them to accommodate different contours and the regularities on the surfaces over which they are applied.

In use, some embodiments of the present invention may include a backing portion which is used to substantially cover and protect the wound dressing and treatment composition. The backing portion is preferably breathable and waterproof or an absorbent material to cope with excess wound exudation.

In use, other embodiments of the present invention may include an outer covering which is used to substantially cover and protect the backing portion, wound dressing and treatment composition. The outer covering can be waterproof and/or of a breathable covering material. For example the outer covering may be from the material sold under the trade mark name of Goretex™.

Protection of the treatment composition may be preferred in dressing systems where the treatment composition must be maintained.

In various embodiments of the present invention both a backing portion and outer covering can be utilised, or alternatively used independently and may be removable from the wound dressing while the dressing is in-situ.

In other embodiments the wound dressing of the present invention may simply be covered by a bandage, or other medical dressing or covering known in the art.

Standard suturing techniques leave scarring through a number of means, including tissue reaction to the suture thread, misalignment of the wound, bunching of the skin and so forth.

Preferably, the wound dressing of the present invention is able to align both sides of the wound in the same plane though the physical pressure of the honey composition upon both sides of the wound, reducing scarring and promoting faster healing.

The honey composition also confers a degree of structural rigidity to the wound dressing, allowing it to be positioned across the wound easily and rapidly, in contrast to dressings such as butterfly closures whose lack of rigidity makes them difficult to apply.

In some preferred embodiments the gelled honey composition may be a layer which substantially covers the outer/non-adhesive side of the adhesive portions in addition to the non-elastic threads. This further increases the rigidity of the dressing and thus the ability to keep both sides of the wound in the same plane.

The extra rigidity also makes placement on skin easier where it is difficult to hold the edges of a wound together until the dressing is stuck to the skin.

The ability of the present invention to hold the wound closed without the use of invasive sutures also eliminates the risk of scarring due to tissue reaction or to the bunching of the skin.

Further, as the threads do not need to be placed in the skin, it is possible to use many more threads than wound normally be used to close a wound. This allows the wound to be held closed at many points, rather than a few, resulting is faster healing and with less scarring than typical suture methods.

As no foreign bodies are inserted into the skin, the wound dressing of the present invention is both easier to place, more comfortable in use and also does not cause scarring as do many existing suture methods.

Further, given the sutured threads are not inserted into the skin, the wound dressing is able to flex in a direction substantially parallel to the wound.

Previously, stretching of the skin around sutures resulted in the pulling on the thread, which is both uncomfortable and painful to the patient.

As the sutured threads are fused to the adhesive strips rather than being inserted into the skin, wounds may be closed without the need for local or general anaesthetic. This is particularly advantageous with abdominal wounds, or when dressing wounds in the field.

Further, the ability of the present invention to be applied without invasive techniques also allow it to find use in veterinary medicine and well as human medicine, especially with non-compliant patients or children.

After the wound has healed, the wound dressing of the present invention may be kept in place, with or without a treatment composition, given that wounds are typically not full strength until up to six months after the injury.

The elimination of the requirement to remove sutures within two weeks allows a degree of tension to be applied while the scar tissue is strengthening, reducing the likelihood of the scar reopening or stretching.

### BRIEF DESCRIPTION OF DRAWINGS

Further aspects of the present invention will become apparent from the following description which is given by way of example only and with reference to the accompanying drawings in which:

FIG. 1 shows a diagrammatic representation of one preferred embodiment of the present invention;

FIG. 2 shows a diagrammatic representation of a second preferred embodiment of the present invention.

### BEST MODES FOR CARRYING OUT THE INVENTION

With reference to the figures, there is provided a wound dressing as indicated by arrow 1.

The wound dressing (1) consists of two adhesive portions (2), between which is held a plurality of non-elastic threads (3).

The threads (3) are preferably monofilament suture threads which are fused or bonded to the adhesive portions (2).

The threads (3) are positioned with respect to the adhesive portions (2) so that when the wound dressing (1) is applied to the skin, tension can be maintained between the adhesive portions (2) by the non-elastic threads (3).

The adhesive portions (2) are capable of adhering to and being removed from skin as required.

The adhesive portions (2) also contain a plurality of elastomer threads (4) which allow the skin to stretch in one direction only without disturbing the tension held across the wound by the non-elastic threads (3).

The non-elastic threads (3) are configured to allow access to the wound between the adhesive portions (2) without disruption. As such, a treatment composition (5) such as gelled honey can be applied to a wound without disturbing the wound dressing (1).

A second preferred embodiment is shown by FIG. 2. In these embodiments the honey composition (5) substantially covers the outer/non-adhesive side of the adhesive portions (2) in addition to the area between the non-elastic threads (3) to increase the rigidity of the wound dressing (1). The extra rigidity also makes placement on skin easier where it is difficult to hold the edges of a wound together until the dressing is stuck to the skin.

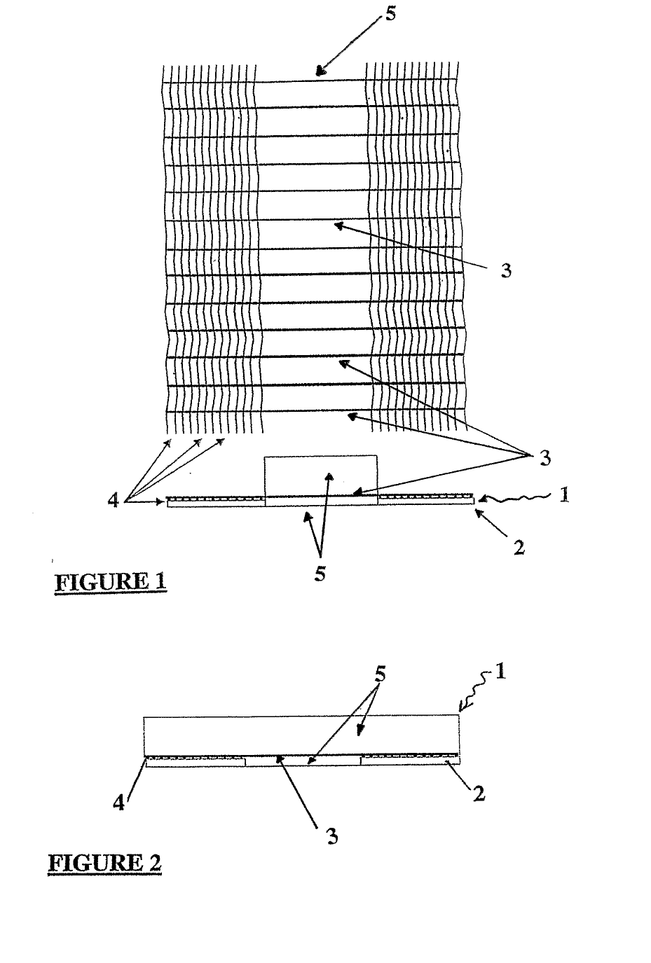

The honey composition is applied as a thin layer (the same thickness as the adhesive strip below the transverse suture thread) on the adhesive side between the two adhesive strips and a thicker layer on the non-adhesive side (between the strips for the version in FIG. 1, and over the whole device for that in FIG. 2).

Pressure on the various components laminate these together in the manufacturing process.

The wound dressing (1) is then covered with a suitable backing portion or standard dressing (not shown) to substantially cover and protect the wound dressing (1) and treatment composition (5).

Aspects of the present invention have been described by way of example only and it should be appreciated that modifications and additions may be made thereto without departing from the scope of the appended claims.

1. A wound dressing that is pre-formed including:
at least two adhesive portions capable of adhering to and being removed from skin, and
at least two non-elastic threads held between the adhesive portions, a treatment composition positioned at least partially between the adhesive portions characterised in that
the spacing between the non-elastic threads allows direct access to the wound or skin between the adhesive portions by the treatment composition.

2. The wound dressing as claimed in claim 1, wherein the non-elastic threads are positioned with respect to the adhesive portions so that tension can be maintained between the adhesive portions by the threads when the dressing is applied to the skin.

3. The wound dressing as claimed in claim 1, wherein the adhesive portions are elastic and can stretch in a direction substantially parallel to that of the wound.

4. The wound dressing as claimed in claims 1, wherein the spacing of the non-elastic threads are within a range of 1-10 mm.

5. The wound dressing as claimed in claim 4, wherein the non-elastic threads are spaced in the order of 5 mm apart from each other.

6. The wound dressing as claimed in claims 1, wherein the treatment composition is an ointment, cream, gel solid or so forth.

7. The wound dressing as claimed in claim 6, wherein the treatment composition configured with the wound dressing is removable.

8. The wound dressing as claimed in claims 6, wherein the treatment composition is a honey composition.

9. The wound dressing as claimed in claim 6, wherein the treatment composition is configured for the wound dressing by incorporation into a pad during manufacture or is applied to a pad itself.

10. The wound dressing as claimed in claims 6, wherein the treatment composition is dissolvable.

11. The wound dressing as claimed in claims 1, which includes a backing portion that substantially wraps over to cover and protects the wound dressing and treatment composition.

12. The wound dressing as claimed in claims 1, which includes an outer covering to substantially cover and protect the backing portion, wound dressing and treatment composition.

13. The wound dressing configured to allow replacement of a treatment composition in the wound dressing as claimed in claims 1.

14-18. (canceled)

19. The wound dressing as claimed in claim 2, wherein the adhesive portions are elastic and can stretch in a direction substantially parallel to that of the wound.

20. The wound dressing as claimed in claim 2, wherein the spacing of the non-elastic threads are within a range of 1 to 10 mm.

21. The wound dressing as claimed in claim 3, wherein the spacing of the non-elastic threads are within a range of 1 to 10 mm.
